(19) United States
(12) Patent Application Publication
(10) Pub. No.: US 2002/0002139 A1
(43) Pub. Date: Jan. 3, 2002

(54) Methods for treating pathologies

(75) Inventors: Thomas W. Hodge, Roswell, GA (US); David W. Boykin, Atlanta, GA (US)

Correspondence Address: Gwendolyn D. Spratt, Esq., NEEDLE & ROSENBERG, P.C., The Candler Building, Suite 1200, 127 Peachtree Street, N.W., Atlanta, GA, 30303-1811, US

(73) Assignee: Omega Pharmaceutical, Inc.

(21) Appl. No.: 09/730,003

(22) Filed: Dec. 4, 2000

Related U.S. Application Data
(63) Continuation of application No. 09/220,052, filed on Dec. 23, 1998, now abandoned.
Application No. 09/220,052 is a continuation of application No. 08/978,685, filed on Nov. 26, 1997, now abandoned.
Application No. 08/978,685 is a continuation of application No. 08/758,976, filed on Dec. 2, 1996, now abandoned.
Application No. 08/758,976 is a continuation of application No. 08/461,717, filed on Jun. 5, 1995, now abandoned.
Application No. 08/461,717 is a continuation of application No. 08/063,218, filed on May 17, 1993, now abandoned.

(51) Int. Cl. A61K 31/7024; A61K 31/216; A61K 31/11

(52) U.S. Cl. 514/25; 514/543; 514/701

(57) ABSTRACT

The invention provides a method of modulating the immune system. Also provided is a method of treating a pathology associated with increased levels of a tumor necrosis factor. The invention further provides a method of treating a pathology resulting from infection by human immunodeficiency virus. Also provided is a method of treating chronic graft vs. host disease. Finally, the invention provides a method of promoting wound healing in a subject.

### BACKGROUND OF THE INVENTION

1. Field of the Invention

This invention relates to the use of purified compounds to treating various pathologies.

2. Background Art

Immune disorders, viral infection and other pathologies continue to pose serious and often fatal conditions in humans. Effective treatments for such pathologies are often ineffective or nonexistent.

A crude extract derived from the root of Eleutherococcus senticosus maxium (E. maxium) has been utilized to treat various disorders. This E. maxium root extract has been marketed in combination with a spleen extract as PCM-4. Although useful, the E. maxium root extraction is laborious and expensive. Further, undesired contaminants exist in extract and consistency of product can be problematic.

Until now, the compounds which produced the varied therapeutic effects remained a mystery. Thus, there exists a great need to identify the compound(s) contained in the E. maxium root extract. The present invention satisfies this need by providing purified compounds having therapeutic activity.

### SUMMARY OF THE INVENTION

The invention provides a method of modulating the immune system. Also provided is a method of treating a pathology associated with increased levels of a tumor necrosis factor. The invention further provides a method of treating a pathology resulting from infection by human immunodeficiency virus. Also provided is a method of treating chronic graft vs. host disease. Finally, the invention provides a method of promoting wound healing in a subject.

These pathologies are treated by administering therapeutic amounts of one or more compounds selected from the group consisting of coniferylaldehyde, caffeic acid ethyl ester, sinapinalcohol, chlorogenic acid, eleutheroside B, eleutheroside B₁, 6,8-dimethoxy-7-hydroxycoumarin, syrinagresinol, eleutheroside E, and eleutheroside A.

### DETAILED DESCRIPTION OF THE INVENTION

The invention provides a method of modulating the immune system. Also provided is a method of treating a pathology associated with increased levels of a tumor necrosis factor. The invention further provides a method of treating a pathology resulting from infection by human immunodeficiency virus and other viruses. Also provided is a method of treating chronic graft vs. host disease and autoimmune diseases. Also provided is a method of preventing or reducing the effects of ethanol on a human subject. Further provided is a method of preventing metastasis. Still further, the invention provides a method for preventing and treating diarrhea. Finally, the invention provides a method of promoting wound healing in a subject.

These pathologies are treated by administering therapeutic amounts of one or more compounds, including derivatives or analogs thereof, having the following structural formula:

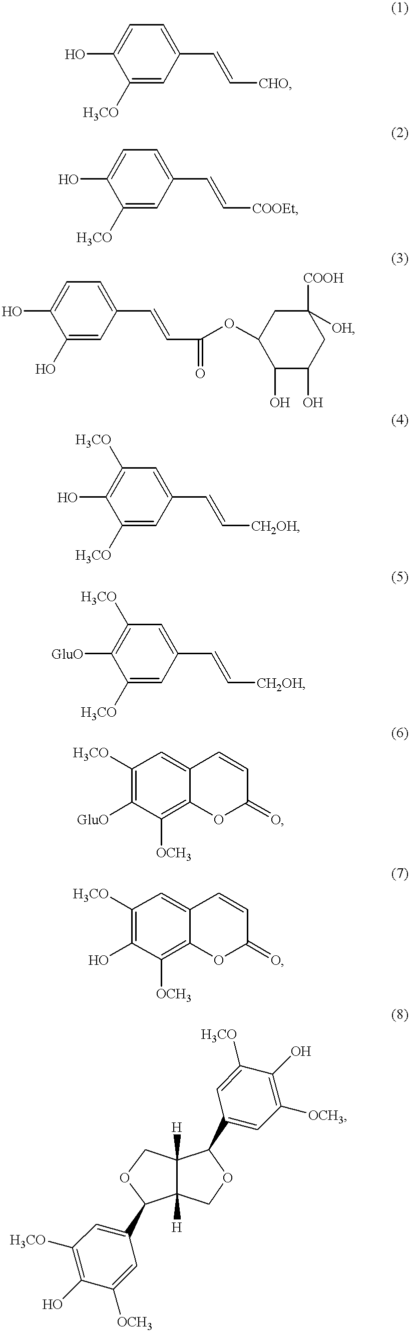

Examples of effective structures including analogs of (4) and (5) are as follows:

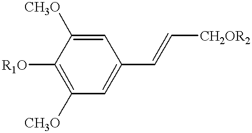

wherein R₁ is selected from galactoside, mannoside, H and glucoside and wherein R₂ is selected from H and CO(CH₂)nCH₃, wherein n=0,1-6.

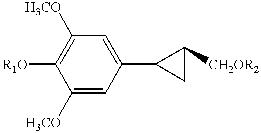

wherein R₁ is selected from galactoside, mannoside, H and glucoside and wherein R₂ is selected from H and CO(CH₂)nCH₃, wherein n=0,1-6.

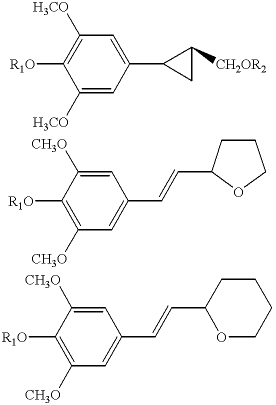

wherein R₁ is selected from the group consisting of H, glucoside, galactoside and mannoside and wherein R₂ is selected from H and CO(CH₂)nCH₃, wherein n=0,1-6.

Examples of effective structures including analogs of (6) and (7) are as follows:

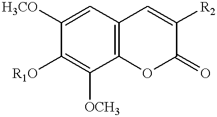

wherein R₁ is selected from galactoside, mannoside, H and glucoside, wherein R₂ is selected from H, Ar, alkyl, wherein

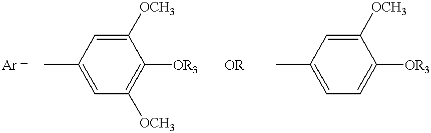

wherein R₃=glucoside, galactoside and mannoside.

Examples of effective structures including analogs of (8) and (9) are as follows:

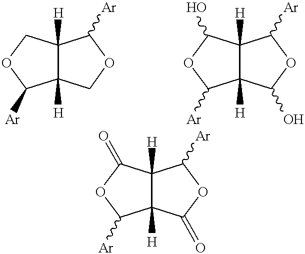

wherein

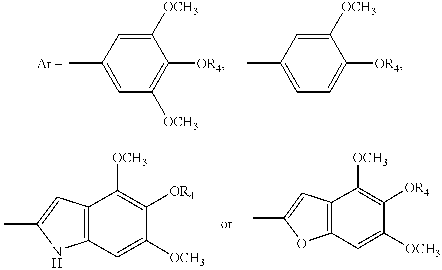

wherein R₄=H, glucoside, mannoside, —CO(CH₂)nCh₃n=0,1-6.

Examples of effective analogs of (10) are as follows:

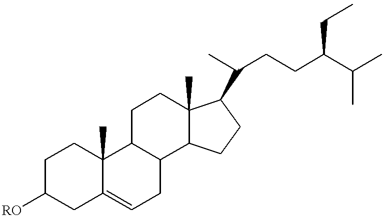

wherein R=glucoside, galactoside, mannoside and CO(CH₂)nCH₃ n=0,1-6.

### Defintions and Nomenclature

It is to be understood that this invention is not limited to specific synthetic methods, specific pharmaceutical carriers, or to particular pharmaceutical formulations or administration regimens, as such may, of course, vary. It is also to be understood that the terminology used herein is for the purpose of describing particular embodiments only and is not intended to be limiting.

It must be noted that, as used in the specification and the appended claims, the singular forms “a,” “an” and “the” can include plural referents unless the context clearly dictates otherwise. Thus, for example, reference to “a compound” includes mixtures of compounds, reference to “a pharmaceutical carrier” includes mixtures of two or more such carriers, and the like.

By the term “therapeutic amount” of a compound as provided herein is meant a nontoxic but sufficient amount of the compound to provide the desired activity. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “therapeutic amount.” However, an appropriate therapeutic amount may be determined by one of ordinary skill in the art using only routine experimentation.

The compounds of the invention can exist in pharmaceutically acceptable carriers. By “pharmaceutically acceptable” is meant a material that is not biologically or otherwise undesirable, i.e., the material may be administered to an individual along with the selected compound without causing any undesirable biological effects or interacting in a deleterious manner with any of the other components of the pharmaceutical composition in which it is contained.

By “modulating” is meant that a compound is capable of acting as an activator/agonist or an antagonist of the immune system.

The invention also encompasses pharmaceutically acceptable nontoxic ester, glycoside, amide and salt derivatives of the compounds.

Pharmaceutically acceptable salts are prepared by treating the free acid with an appropriate amount of pharmaceutically acceptable base. Representative pharmaceutically acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. The reaction is conducted in water, alone or in combination with an inert, water-miscible organic solvent, at a temperature of from about 0° C. to about 100° C., preferably at room temperature. The molar ratio of the compounds to base used are chosen to provide the ratio desired for any particular salts. For preparing, for example, the ammonium salts of the free acid starting material—a particular preferred embodiment herein—the starting material can be treated with approximately one equivalent of pharmaceutically acceptable base to yield a neutral salt. When calcium salts are prepared, approximately one-half a molar equivalent of base is used to yield a neutral salt, while for aluminum salts, approximately one-third a molar equivalent of base will be used.

Ester derivatives are typically prepared as precursors to the acid form of the compounds and accordingly may serve as prodrugs. Generally, these derivatives will be lower alkyl esters such as acetate, propionate, and the like. Amide derivatives, —(CO)NH₂, —(CO)NHR and —(CO)NR₂, where R is lower alkyl, may be prepared by the reaction of the carboxylic acid compound by activation and subsequent treatment with ammonia or a substituted amine.

The invention also encompasses pharmaceutically acceptable nontoxic analogs of the compounds. Compounds possessing substantially the same properties as the described compounds and which are equivalents thereof are those bearing one or more simple substituents, including, for example, lower alkyl e.g., methyl, ethyl, butyl; halo, e.g., chloro, fluoro, bromo; nitro; sulfato; sulfonyloxr, carboxy, carbo-lower-alkoxy, e.g., carbomethoxy, carbethoxy; amino; mono-and di-lower-alkylamino, e.g., methyl-amino, methylamino, dimethylamino, methylethylamino; amido; hydroxy; lower-alkoxy, e.g., methoxy, ethoxy, and lower-alkanoyloxy, e.g., acetoxy.

### Synthetic Methods

The compounds of the invention may be readily synthesized using techniques generally known to synthetic organic chemists. Suitable experimental methods for making and derivatizing compounds are described in the art.

Methods for making specific and preferred compounds of the present invention are described in detail below.

### Utility and Administration

The compounds of the invention defined by structural formula, including the pharmacologically acceptable analogs, esters, amides or salts thereof, are useful to elicit a therapeutic response.

The compounds of the invention may be conveniently formulated into pharmaceutical compositions composed of one or more of the compounds in association with a pharmaceutically acceptable carrier. See, e.g., Remington's Pharmaceutical Sciences. latest edition, by E. W. Martin (Mack Publ. Co., Easton Pa.) discloses typical carriers and conventional methods of preparing pharmaceutical compositions that may be used in conjunction with the preparation of formulations of the compounds.

The compounds may be administered orally, parenterally (e.g., intravenously), by intramuscular injection, by intraperitoneal injection, topically, transdermally, or the like, although oral or topical administration is typically preferred. The amount of active compound administered will, of course, be dependent on the subject being treated, the subject's weight, the manner of administration and the judgment of the prescribing physician. Generally, however, dosage will approximate that which is typical for the administration of like compounds.

Depending on the intended mode of administration, the pharmaceutical compositions may be in the form of solid, semi-solid or liquid dosage forms, such as, for example, tablets, suppositories, pills, capsules, powders, liquids, suspensions, lotions, creams, gels, or the like, preferably in unit dosage form suitable for single administration of a precise dosage. The compositions will include, as noted above, an effective amount of the selected drug in combination with a pharmaceutically acceptable carrier and, in addition, may include other medicinal agents, pharmaceutical agents, carriers, adjuvants, diluents, etc.

For solid compositions, conventional nontoxic solid carriers include, for example, pharmaceutical grades of mannitol, lactose, starch, magnesium stearate, sodium saccharin, talc, cellulose,. glucose, sucrose, magnesium carbonate, and the like. Liquid pharmaceutically administrable compositions can, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; for example, see Remington's Pharmaceutical Sciences, referenced above.

For oral administration, fine powders or granules may contain diluting, dispersing, and/or surface active agents, and may be presented in water or in a syrup, in capsules or sachets in the dry state, or in a nonaqueous solution or suspension-wherein suspending agents may be included, in tablets wherein binders and lubricants may be included, or in a suspension in water or a syrup. Where desirable or necessary, flavoring, preserving, suspending, thickening, or emulsifying agents may be included. Tablets and granules are preferred oral administration forms, and these may be coated.

Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system, such that a constant level of dosage is maintained. See, e.g., U.S. Pat. No. 3,710,795, which is incorporated by reference herein.

### EXPERIMENTAL

The following examples are put forth so as to provide those of ordinary skill in the art with a complete disclosure and description of how the compounds claimed herein are made and evaluated, and are not intended to limit the scope of what the inventors regard as their invention. Efforts have been made to ensure accuracy with respect to numbers (e.g., amounts, temperature, etc.) but some errors and deviations should be accounted for.

### EXAMPLE I

Coniferylaldehyde (1), caffeic acid (2), and chlorogenic acid (3) are commercially available (Aldrich Chemical Co.).

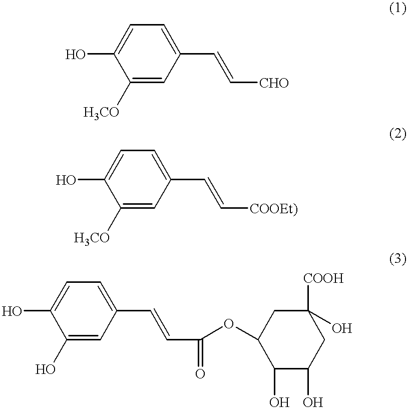

These compounds, and their derivatives or analogs, can be administered to a subject to produce one or more therapeutic effects including modulation of the immune system, treatment of a pathology resulting from infection by human immunodeficiency virus, treatment of chronic graft vs. host disease, treatment of autoimmune disease, antiviral activities, prevention or reduction of the effects of ethanol, prevention of metastasis, prevention and treatment of toxic shock, diarrhea and promotion of wound healing.

### EXAMPLE II

Sinapinalcohol (4) can be synthesized as follows:

3,5-dimethoxy4-hydroxycinnamic acid (Aldrich Chemical Co.) is reacted with acetic anhydride to produce 3,5-dimethoxy4-acetoxycinnamic acid. The acetoxy derivative is reacted with thionyl chloride and treated with ethanol to yield its ethyl ester. The ethyl ester of 3,5-dimethoxy4-acetoxycinnamic acid is then reduced with lithium aluminum hydride in ether under nitrogen at −15° C. according to the method of Freudenberg and Dillenburg (Chem. Ber. 84:67 (1951)).

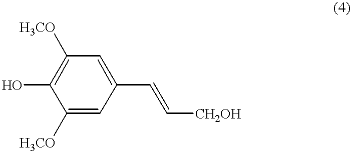

This compound, and its derivatives or analogs, can be administered to a subject to produce one or more therapeutic effects including modulation of the immune system, treatment of a pathology resulting from infection by human immunodeficiency virus, treatment of chronic graft vs. host disease, treatment of autoimmune disease, antiviral activities, prevention or reduction of the effects of ethanol, prevention of metastasis, prevention and treatment of toxic shock, diarrhea and promotion of wound healing.

### EXAMPLE III

Eleutheroside B (5) is obtained by reaction of sinapinalcohol (4) with α-D-glucopyranosyl bromide tetraacetate in acetone solution in the presence of sodium hydroxide at room temperature. The acetate groups are removed by the action of NaOCH₃/CH₃OH at room temperature, the solution is made neutral with acetic acid, the solvent was evaporated and the resulting solid is purified by crystalization from ethanol.

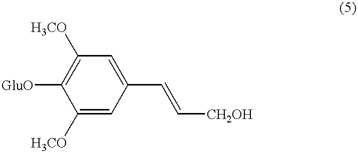

This compound, and its derivatives or analogs, can be administered to a subject to produce one or more therapeutic effects including modulation of the immune system, treatment of a pathology resulting from infection by human immunodeficiency virus, treatment of chronic graft vs. host disease, treatment of autoimmune disease, antiviral activities, prevention or reduction of the effects of ethanol, prevention of metastasis, prevention and treatment of toxic shock, diarrhea and promotion of wound healing.

### EXAMPLE IV

Eleutheroside B₁ is obtained by reacting 6,8-dimethoxy-7-hydroxycoumarin (isofraxidin) (7) (see Example V) in the presence of base with α-D-glucopyranosyl bromide tetra acetate followed by hydrolysis of the acetate groups as described for (5).

Alternatively, Eleutheroside B₁ (5) can be obtained as follows. 7-acetylcoumarin (Aldrich Chemical Co.) is heated with aluminum chloride at 165 C. for 1 hr and yields on cooling to room temperature a solid mass which is treated first with HCl and then extracted with 2 M NaOH, the latter solution on acidification gives 8-acetyl-7-hydroxycoumarin (Limaye and Joshi, Rosayanam 1:225 (1941)). The latter compound reacts with benzyl bromide in acetone in the presence of potassium carbonate to give 8-acetyl-7-benzyloxycoumarin. The latter compound is dissolved in 15% potassium hydroxide by heating near 100 C. for 1 hr; the solution is cooled to 10 C. and then treated with 5% potassium persulphate and stirred for 6 hr. at 10 C. and then 24 hr. at room temperature. The solution is acidified to near pH 5 and unreacted coumarin is removed by filtration. The filtrate is acidified with concentrated HCl and heated at 80 C. for 2 hr. The solid which is collected is 8-acetyl-7-benzyloxy6-hydroxycoumarin. The latter compound on treatment with methyl sulfate in acetone in the presence of potassium carbonate yields 8-acetyl-7-benzyloxy-6-methoxycoumarin. The latter compound is treated with 1 M NaOH and 30% hydrogen peroxide at 0 C.; the temperature of the mixture is allowed to come to room temperature and after an hour the crystalline 8-hydroxy-7-benzyloxy-6-methoxycoumarin is filtered. Treatment of the latter compound with methyl sulfate as above gives 6,8-dimethoxy-7-benzyloxycoumarin. The benzyl group is removed with Pd/C in cyclohexene and the resulting 6,8-dimethoxy-7-hydroxycoumarin is treated with α-D-glucopyranoysyl bromide tetra acetate as described for (5) to yield Eleutheroside B₁.

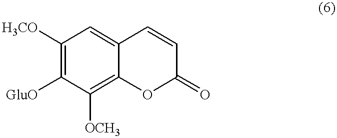

This compound, and its derivatives or analogs, can be administered to a subject to produce one or more therapeutic effects including modulation of the immune system, treatment of a pathology resulting from infection by human immunodeficiency virus, treatment of chronic graft vs. host disease, treatment of autoimmune disease, antiviral activities, prevention or reduction of the effects of ethanol, prevention of metastasis, prevention and treatment of toxic shock, diarrhea and promotion of wound healing.

Confusion exists in the literature regarding the stereochemistry of (6). The compound called Eleutheroside B₁ is represented as an α-glycoside (Chem. Abst. 67:54394 (1967)); the compound called calycanthoside is represented as the β-glycoside (Bull. Soc. Chem. Biol. 37:365 (1955); Hebd. Seances Acad, Sci. 226D:1763 (1968)). Both compounds, if different, are glycosides of (7) (isofraxidin). The β compound is reported in Chem. Pharm. Bull. 38:1763 (1990), where it is referred to only by the IUPAC name. Thus, both isomers can be synthesized and utilized as appropriate.

### EXAMPLE V

The method of Spath et al. (Chem. Ber. 70:1672 (1937)) is employed to prepare 7 by reacting commercially available 7,8-dihydroxy-6-methoxycoumarin (fraxetin) (Aldrich Chemical Co.) with one equivalent of diazomethane in ether to give a mixture of isofraxidin (7) and fraxidin which is separated by distillation followed by fractional crystallization or by chromatography.

An alternative synthesis of 6,8-dimethoxy-7-hydroxycoumarin is described in Example III.

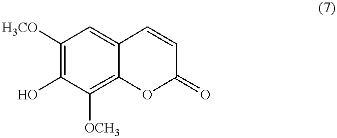

This compound, and its derivatives or analogs, can be administered to a subject to produce one or more therapeutic effects including modulation of the immune system, treatment of a pathology resulting from infection by human immunodeficiency virus, treatment of chronic graft vs. host disease, treatment of autoimmune disease, antiviral activities, prevention or reduction of the effects of ethanol, prevention of metastasis, prevention and treatment of toxic shock, diarrhea and promotion of wound healing.

### EXAMPLE VI

Syringaresinol (8) is prepared by the method of Pelter et al. (J. Chem. Soc. Perkin 1:175 (1982)) starting by oxidative coupling of 3,5-dimethoxy-4-hydroxycinnamic acid with ferric chloride to form 4,8-[3,5dimethoxy-4-hydroxyphenyl]-3,7-dioxabicyclo[3.3.0]octane-2,6-dione. The bis-lactone is reduced with DIBAL at low temperature to yield the dilactol 4,8-[3,5-dimethoxy-4-hydroxyphenyl]-3,7dioxabicyclo[3.3.0]octane-3,6-diol. Treatment of the diol with toluene-p-sulphonyl chloride in pyridine gives a tetratosylate which is reduced with lithium aluminum hydride in tetrahydrofuran to yield 8.

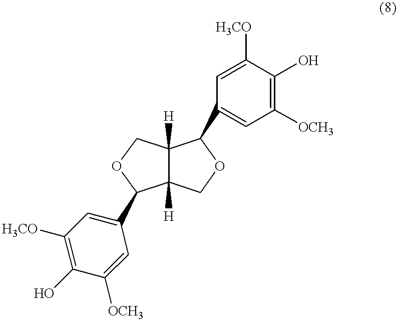

This compound, and its derivatives or analogs, can be administered to a subject to produce one or more therapeutic effects including modulation of the immune system, treatment of a pathology resulting from infection by human immunodeficiency virus, treatment of chronic graft vs. host disease, treatment of autoimmune disease, antiviral activities, prevention or reduction of the effects of ethanol, prevention of metastasis, prevention and treatment of toxic shock, diarrhea and promotion of wound healing.

### EXAMPLE VII

Syringaresinol (8) yields Eleutheroside E (9) on reaction with α-D-glucopyranosyl bromide tetraacetate followed by removal of the acetate groups by basic hydrolysis as described in (5).

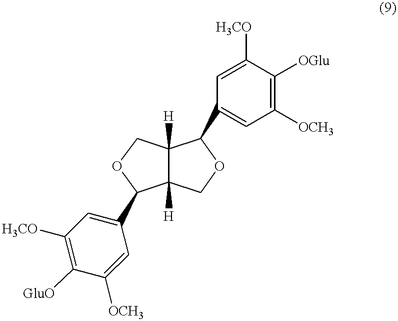

This compound, and its derivatives or analogs, can be administered to a subject to produce one or more therapeutic effects including modulation of the immune system, treatment of a pathology resulting from infection by human immunodeficiency virus, treatment of chronic graft vs. host disease, treatment of autoimmune disease, antivial activities, prevention or reduction of the effects of ethanol, prevention of metastasis, prevention and treatment of toxic shock diarrhea and promotion of wound healing.

### EXAMPLE VIII

B-Sitosterol (ICN Biomedicals) reacts with α-D-glucopyranosyl bromide tetra acetate in acetone in the presence of sodium hydroxide at room temperature to yield, after removal of the acetate groups with NaOCH₃/CH₃OH as described for (5). (T. Kiribuchi, et al., Agr. Biol. Chem. 31:1244 (1967)).

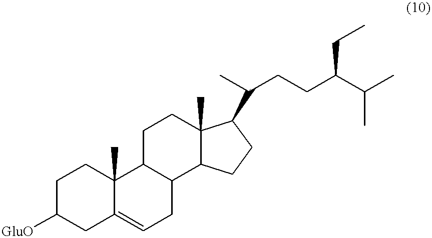

This compound, and its derivatives or analogs, can be administered to a subject to produce one or more therapeutic effects including modulation of the immune system, treatment of a pathology resulting from infection by human immunodeficiency virus, treatment of chronic graft vs. host disease, treatment of autoimmune disease, antiviral activities, prevention or reduction of the effects of ethanol, prevention of metastasis, prevention and treatment of toxic shock, diarrhea and promotion of wound healing.

What is claimed is:

1. A method of modulating the immune system in a subject comprising administering to the subject a therapeutic amount of the compound having the structural formula 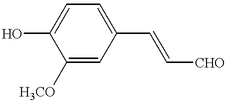
and derivatives or analogs thereof.

2. A method of modulating the immune system in a subject comprising administering to the subject a therapeutic amount of the compound having the structural formula 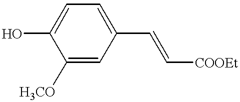
and derivatives or analogs thereof.

3. A method of modulating the immune system in a subject comprising administering to the subject a therapeutic amount of the compound having the structural formula 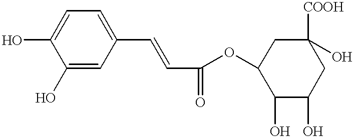
and derivatives or analogs thereof.

4. A method of modulating the immune system in a subject comprising administering to the subject a therapeutic amount of the compound having the structural formula 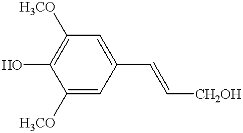
and derivatives or analogs thereof.

5. A method of modulating the immune system in a subject comprising administering to the subject a therapeutic amount of the compound having the structural formula 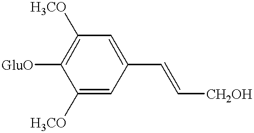
and derivatives or analogs thereof.

6. A method of modulating the immune system in a subject comprising administering to the subject a therapeutic amount of the compound having the structural formula 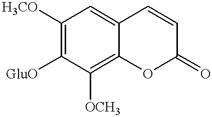
and derivatives or analogs thereof.

7. A method of modulating the immune system in a subject comprising administering to the subject a therapeutic amount of the compound having the structural formula 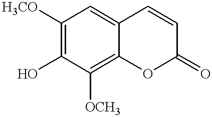
and derivatives or analogs thereof.

8. A method of modulating the immune system in a subject comprising administering to the subject a therapeutic amount of the compound having the structural formula 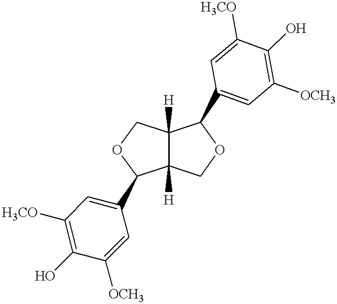
and derivatives or analogs thereof.

9. A method of modulating the immune system in a subject comprising administering to the subject a therapeutic amount of the compound having the structural formula:and derivatives or analogs thereof. 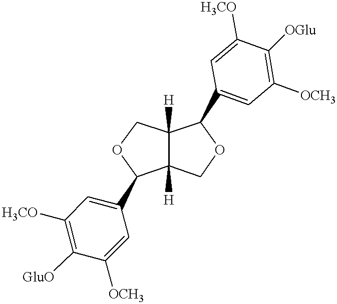
and derivatives or analogs thereof.

10. A method of modulating the immune system in a subject comprising administering to the subject a therapeutic amount of the compound having the structural formula:and derivatives or analogs thereof. 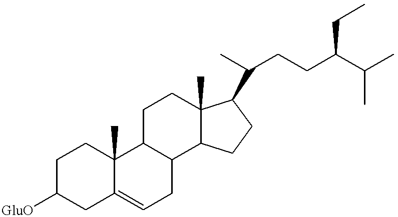
and derivatives or analogs thereof.

11. A method of treating a pathology associated with increased levels of a tumor necrosis factor comprising administering to the subject a compound having the structural formula

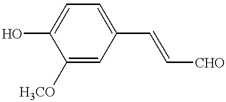

12. A method of treating a pathology associated with increased levels of a tumor necrosis factor comprising administering to the subject a compound having structural formula

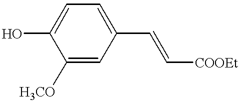

13. A method of treating a pathology associated with increased levels of a tumor necrosis factor comprising administering to the subject a compound having the structural formula

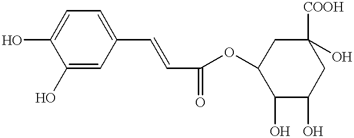

14. A method of treating a pathology associated with increased levels of a tumor necrosis factor comprising administering to the subject a compound having the structural formula

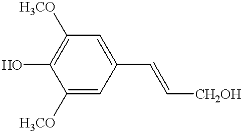

15. A method of treating a pathology associated with increased levels of a tumor necrosis factor comprising administering to the subject a compound having the structural formula

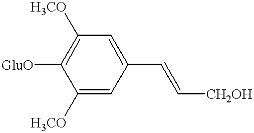

16. A method of treating a pathology associated with increased levels of a tumor necrosis factor comprising administering to the subject a compound having the structural formula

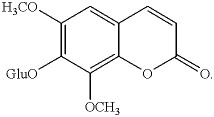

17. A method of treating a pathology associated with increased levels of a tumor necrosis factor comprising administering to the subject a compound having the structural formula

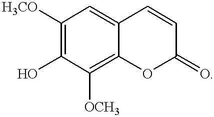

18. A method of treating a pathology associated with increased levels of a tumor necrosis factor comprising administering to the subject a compound having the structural formula

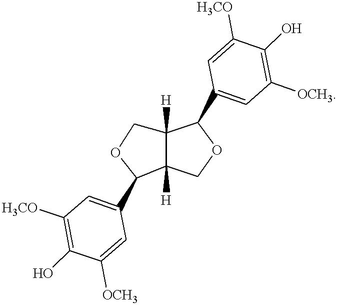

19. A method of treating a pathology associated with increased levels of a tumor necrosis factor comprising administering to the subject a compound having the structural formula

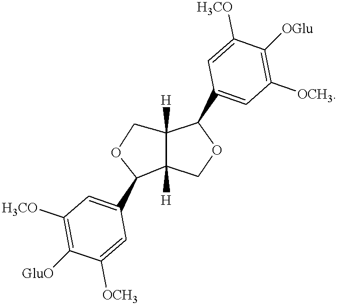

20. A method of treating a pathology associated with increased levels of a tumor necrosis factor comprising administering to the subject a compound having the structural formula

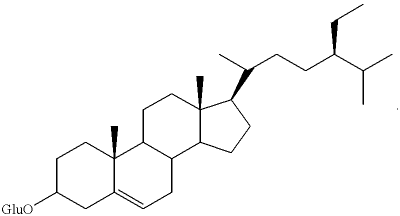

21. A method of treating a pathology resulting from infection by human immunodeficiency virus in a human subject comprising administering to the subject a therapeutic amount of the compound having the structural formula 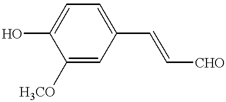
and derivatives or analogs thereof.

22. A method of treating a pathology resulting from infection by human immunodeficiency virus in a human subject comprising administering to the subject a therapeutic amount of the compound having the structural formula 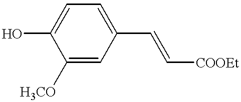
and derivatives or analogs thereof.

23. A method of treating a pathology resulting from infection by human immunodeficiency virus in a human subject comprising administering to the subject a therapeutic amount of the compound having the structural formula 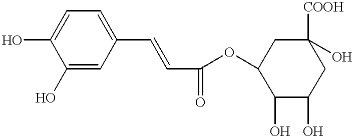
and derivatives or analogs thereof.

24. A method of treating a pathology resulting from infection by human immunodeficiency virus in a human subject comprising administering to the subject a therapeutic amount of the compound having the structural formula 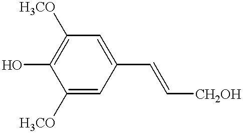
and derivatives or analogs thereof.

25. A method of treating a pathology resulting from infection by human immunodeficiency virus in a human subject comprising administering to the subject a therapeutic amount of the compound having the structural formula 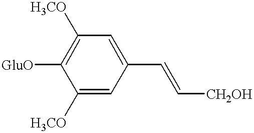
and derivatives or analogs thereof.

26. A method of treating a pathology resulting from infection by human immunodeficiency virus in a human subject comprising administering to the subject a therapeutic amount of the compound having the structural formula 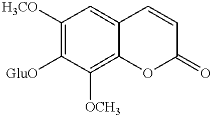
and derivatives or analogs thereof.

27. A method of treating a pathology resulting from infection by human immunodeficiency virus in a human subject comprising administering to the subject a therapeutic amount of the compound having the structural formula 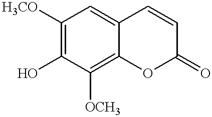
and derivatives or analogs thereof.

28. A method of treating a pathology resulting from infection by human immunodeficiency virus in a human subject comprising administering to the subject a therapeutic amount of the compound having the structural formula 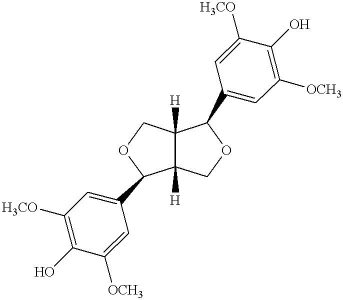
and derivatives or analogs thereof.

29. A method of treating a pathology resulting from infection by human immunodeficiency virus in a human subject comprising administering to the subject a therapeutic amount of the compound having the structural formula 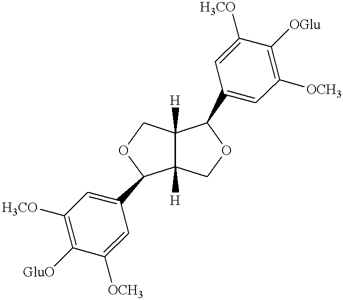
and derivatives or analogs thereof.

30. A method of treating a pathology resulting from infection by human immunodeficiency virus in a human subject comprising administering to the subject a therapeutic amount of the compound having the structural formula 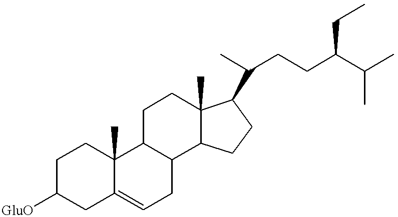
and derivatives or analogs thereof.
